(19) United States
(12) Patent Application Publication
(10) Pub. No.: US 2005/0080139 A1
(43) Pub. Date: Apr. 14, 2005

(54) Naaladase inhibitors for treating huntington's disease

(76) Inventors: Barbara S. Slusher, Kingsville, MD (US); Krystyna Wozniak, Bel Air, MD (US)

Correspondence Address: GUILFORD PHARMACEUTICALS C/O;FOLEY & LARDNER, 3000 K STREET, NW, WASHINGTON, DC, 20007-5143, US

(21) Appl. No.: 10/500,323

(22) PCT Filed: Dec. 27, 2002
(86) PCT No.: PCT/US02/41571

(30) Foreign Application Priority Data
Dec. 28, 2001 (US) 60342770

(51) Int. Cl. A61K 31/19

(52) U.S. Cl. 514/568

(57) ABSTRACT

This invention relates to a pharmaceutical composition and a method for treating Huntington's disease using NAAL-ADase inhibitors.

This application claims the benefit of U.S. application Ser. No. 60/342,770, which is incorporated herein by reference in its entirety.

This invention relates to a pharmaceutical composition and a method for treating Huntington's disease (“HD”) using NAALADase inhibitors.

HD is an inherited neurodegenerative disease associated with severe degeneration of basal ganglia/caudate neurons. Symptoms usually appear in an affected individual at around thirty to fifty years of age and may include unsteady gait, involuntary movements, speech and swallowing difficulties, personality and cognitive changes, depression and mood swings. At present, no treatment is available.

Glutamate has been implicated in the pathophysiology of HD. Studies have reported enhanced NMDA sensitivity (Levine et al., J. Neurosci. Res., Vol. 58, pp. 515 532 (1999), reduced metabotropic GluR (mGluR1, 2 and 3) (Cha et al., PNAS, Vol. 95, pp. 6480-6485 (1998)), and decreased sensitivity to K+-stimulated glutamate release (Nicniocaill et al., Eur. J. Neurosci., Vol. 13, pp. 206-210 (2001)) in HD transgenic mice. Elevated glutamine levels have also been detected in the brains of transgenic HD mice and are believed to result from a decrease in neuronal-glial glutamate-glutamine cycling and a decrease in glutaminase activity (Jenkins et al., J. Neurochem., Vol. 74, pp. 2108-2119 (2000)). It has been proposed that excessive stimulation of glutamate receptors by glutamine may lead to HD (Fischer, Med. Hypotheses, Vol. 48, pp. 393-398 (1997). As further evidence of glutamate's involvement in HD, NMDA agonist quinolinic acid has been shown to cause HD-like lesions (Beal et al., Nature, Vol. 321, pp. 168-171 (1986), while NMDA antagonists have been found to decrease neuronal injury from the mitochondrial toxin 3NPA which causes HD-like neurotoxicity (Ikonomidou et al., PNAS, Vol. 97, pp. 12885-12890 (2000)).

One source of glutamate is derived from the neuropeptide N-acetylated-aspartyl-glutamate (NAAG) through cleavage by N-acetylated-α-linked acidic dipeptidase (NAALADase), also known as prostate specific membrane antigen (PSM or PSMA) and human glutamate carboxypeptidase II (GCP II). Studies suggest that NAALADase inhibitors may block glutamate release pre-synaptically without interacting with post-synaptic glutamate receptors.

This invention relates' to a method for treating Huntington's disease comprising administering an effective amount of a NAALADase inhibitor to a mammal in need of such treatment.

This invention also relates to a pharmaceutical composition comprising:
(i) an effective amount of a NAALADase inhibitor for treating Huntington's disease; and
(ii) a pharmaceutically acceptable carrier.

### BRIEF DESCRIPTION OF THE DRAWINGS

FIG. 1 is bar graph comparing the rotarod performance of transgenic HD mice and normal non-HD mice treated with 2-(3-sulfanylpropyl)-pentanedioic acid (“Compound B”), and transgenic HD mice and normal non-HD mice treated with a vehicle.

FIG. 2 is a bar graph comparing the total distance traveled by transgenic HD mice and normal non-HD mice treated with Compound B, and transgenic HD mice and normal non-HD mice treated with a vehicle.

FIG. 3 is a graph plotting the survival time of transgenic HD mice treated with Compound B or a vehicle.

FIG. 4 is a graph plotting the survival time of male transgenic HD mice treated with Compound B or a vehicle.

FIG. 5 is a graph plotting the survival time of female transgenic HD mice treated with Compound B or a vehicle.

“Compound B” refers to 2-(3-sulfanylpropyl)-pentanedioic acid.

“Alkyl” refers to a branched or unbranched saturated hydrocarbon chain comprising a designated number of carbon atoms. For example, C₁-C₉ alkyl is a straight or branched hydrocarbon chain containing 1 to 9 carbon atoms, and includes but is not limited to substituents such as methyl, ethyl, propyl, iso-propyl, butyl, iso-butyl, tert-butyl, n-pentyl, n-hexyl, and the like, unless otherwise indicated.

“Alkenyl” refers to a branched or unbranched unsaturated hydrocarbon chain comprising a designated number of carbon atoms. For example, C₂-C₉ alkenyl is a straight or branched hydrocarbon chain containing 2 to 9 carbon atoms having at least one double bond, and includes but is not limited to substituents such as ethenyl, propenyl, iso-propenyl, butenyl, iso-butenyl, tert-butenyl, n-pentenyl, n-hexenyl, and the like, unless otherwise indicated.

“Alkoxy” refers to the group —OR wherein R is alkyl as herein defined. In some embodiments, R is a branched or unbranched saturated hydrocarbon chain containing 1 to 9 carbon atoms.

“Carbocycle” refers to a hydrocarbon, cyclic moiety having one or more closed ring(s) that is/are alicyclic, aromatic, fused and/or bridged. Examples include cyclopropane, cyclobutane, cyclopentane, cyclohexane, cycloheptane, cyclopentene, cyclohexene, cycloheptene, cycloctene, benzyl, naphthene, anthracene, phenanthracene, biphenyl and pyrene.

“Aryl” refers to an aromatic, hydrocarbon cyclic moiety having one or more closed ring(s). Examples include, without limitation, phenyl, naphthyl, anthracenyl, phenanthracenyl, biphenyl and pyrenyl.

“Heterocycle” refers to a cyclic moiety having one or more closed ring(s) that is/are alicyclic, aromatic, fused and/or bridged, with one or more heteroatom(s) (for example, sulfur, nitrogen or oxygen) in at least one of the rings. Examples include, without limitation, pyrrolidine, pyrrole, thiazole, thiophene, piperidine, pyridine, isoxazolidine and isoxazole.

“Heteroaryl” refers to an aromatic, cyclic moiety having one or more closed ring(s) with one or more heteroatom(s) (for example, sulfur, nitrogen or oxygen) in at least one of the rings. Examples include, without limitation, pyrrole, thiophene, pyridine and isoxazole.

“Linking group” refers to a moiety that connects the terminal group with the benzene ring in the compounds of formula V, without compromising with the pharmacological or biological activity of the overall compound. A “terminal group” is any group capable of bonding with W or the phenyl of formula V below.

“Metal binding group” refers to a functional group capable of interacting with metal ion(s), such as Co²⁺, Ni²⁺, Mn²⁺, Cu²⁺, Zn²⁺, Mg²⁺, Fe²⁺, Fe³⁺, or Al³⁺, Common metal binding groups include amines (e.g. ethylenediamine), aldehydes, ketones, carboxylic acids (e.g. ethylenediaminetetraacetic acid (EDTA)), thiols, phosphorus derivatives and hydroxamic acids.

“Derivative” refers to a substance produced from another substance either directly or by modification or partial substitution.

“Effective amount” refers to the amount required to produce the desired effect.

“Halo” refers to at least one fluoro, chloro, bromo or iodo moiety.

“Isosteres” refer to elements, functional groups, substitutents, molecules or ions having different molecular formulae but exhibiting similar or identical physical properties. For example, tetrazole is an isostere of carboxylic acid because it mimics the properties of carboxylic acid even though they both have different molecular formulae. Typically, two isosteric molecules have similar or identical volumes and shapes. Ideally, isosteric compounds should be isomorphic and able to co-crystallize. Other physical properties that isosteric compounds usually share include boiling point, density, viscosity and thermal conductivity. However, certain properties are usually different: dipolar moments, polarity, polarization, size and shape since the external orbitals may be hybridized differently. The term “isosteres” encompass “bioisosteres”.

“Bioisosteres” are isosteres that, in addition to their physical similarities, share some common biological properties. Typically, bioisosteres interact with the same recognition site or produce broadly similar biological effects.

“Carboxylic acid isosteres” include without limitation direct derivatives such as hydroxamic acids, acyl-cyanamides and acylsulfonamides; planar acidic heterocycles such as tetrazoles, mercaptoazoles, sulfinylazoles, sulfonylazoles, isoxazoles, isothiazoles, hydroxythiadiazoles and hydroxychromes; and nonplanar sulfur- or phosphorus-derived acidic functions such as phosphinates, phosphonates, phosphonamides, sulphonates, sulphonamides, and acylsulphonamides.

“Metabolite” refers to an intermediate or product resulting from metabolism.

“NAAG” refers to N-acetyl-aspartyl-glutamate, an important peptide component of the brain, with levels comparable to the major inhibitor neurotransmitter gamma-aminobutyric acid (GABA). NAAG is neuron-specific, present in synaptic vesicles and released upon neuronal stimulation in several systems presumed to be glutamatergic. Studies suggest that NAAG may function as a neurotransmitter and/or neuromodulator in the central nervous system, or as a precursor of the neurotransmitter glutamate. In addition, NAAG is an agonist at group II metabotropic glutamate receptors, specifically mGluR3 receptors; when attached to a moiety capable of inhibiting NAALADase, it is expected that metabotropic glutamate receptor ligands will provide potent and specific NAALADase inhibitors.

“NAALADase” refers to N-acetylated a-linked acidic dipeptidase, a membrane bound metallopeptidase that catabolizes NAAG to N-acetylaspartate (“INAA”) and glutamate (“GLU”):

### Catabolism of NAAG by NAALADase

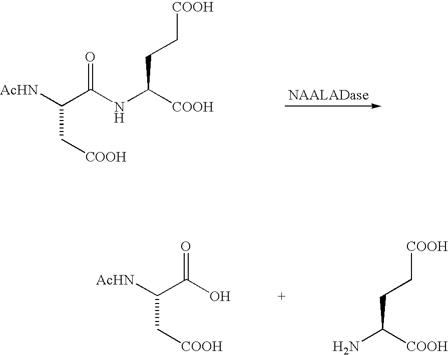 NAALADase has been assigned to the M28 peptidase family and is also called prostate specific membrane antigen (PSM) or human glutamate carboxypeptidase II (GCP II), EC number 3.4.17.21. It is believed that NAALADase is a co-catalytic zinc/zinc metallopeptidase. NAALADase shows a high affinity for NAAG with a Km of 540 nM. If NAAG is a bioactive peptide, then NAALADase may serve to inactivate NAAG'S synaptic action. Alternatively, if NAAG functions as a precursor for glutamate, the primary function of NAALADase may be to regulate synaptic glutamate availability.

“Pharmaceutically acceptable carrier” refers to any carrier, diluent, excipient, wetting agent, buffering agent, suspending agent, lubricating agent, adjuvant, vehicle, delivery system, emulsifier, disintegrant, absorbent, preservative, surfactant, colorant, flavorant, or sweetener, which in some embodiments are non-toxic, that would be suitable for use in a pharmaceutical composition.

“Pharmaceutically acceptable equivalent” includes, without limitation, pharmaceutically acceptable salts, hydrates, metabolites, prodrugs, and isosteres. Many pharmaceutically acceptable equivalents are expected to have the same or similar in vitro or in vivo activity as the inventive compounds.

“Pharmaceutically acceptable salt” refers to a salt of the inventive compounds that possesses the desired pharmacological activity and that is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycero-phosphate, hemisulfate, heptanoate, hexanoate, hydro-chloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. The basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using methods known in the art, such as those described, by Burger's Medicinal Chemistry and Drug Chemistry, Fifth Ed., Vol. 1, pp. 172-178, 949-982 (1995).

“Inhibition,” in the context of enzymes, refers to reversible enzyme inhibition such as competitive, uncompetitive and non-competitive inhibition. Competitive, uncompetitive and non-competitive inhibition can be distinguished by the effects of an inhibitor on the reaction kinetics of an enzyme. Competitive inhibition occurs when the inhibitor combines reversibly with the enzyme in such a way that it competes with a normal substrate for binding at the active site. The affinity between the inhibitor and the enzyme may be measured by the inhibitor constant, Kᵢ, which is defined as:

$${K}_{i} = \frac{[ E ] [ I ]}{[ EI ]}$$

wherein [E] is the concentration of the enzyme, [I] is the concentration of the inhibitor, and [EI] is the concentration of the enzyme-inhibitor complex formed by the reaction of the enzyme with the inhibitor. Unless otherwise specified, Kᵢ as used herein refers to the affinity between the inventive compounds and NAALADase. “IC₅₀” is a related term used to define the concentration or amount of a compound that is required to cause a 50% inhibition of the target enzyme.

“NAALADase inhibitor” refers to any compound that inhibits NAALADase enzyme activity. In some embodiments, a NAALADase inhibitor exhibits a Kᵢ of less than 100 μM, and in some embodiments less than 10 μM, and in some embodiments less than 1 μM, as determined using any appropriate assay known in the art.

“Isomers” refer to compounds having the same number and kind of atoms, and hence the same molecular weight, but differing in respect to the arrangement or configuration of the atoms.

“Optical isomers” refer to enantiomers or diastereoisomers.

“Stereoisomers” are isomers that differ only in the arrangement of the atoms in space.

“Diastereoisomers” are stereoisomers that are not mirror images of each other. Diastereoisomers occur in compounds having two or more asymmetric carbon atoms; thus, such compounds have 2ⁿ optical isomers, where n is the number of asymmetric carbon atoms.

“Enantiomers” are a pair of stereoisomers that are non-superimposable mirror images of each other. Enantiomers result, for example, from the presence of one or more asymmetric carbon atom(s) in the compound (e.g., glyceraldehyde, lactic acid, sugars, tartaric acid, amino acids).

“Enantiomer-enriched” refers to a mixture in which one enantiomer predominates.

“Racemic mixture” means a mixture containing equal amounts of enantiomers.

“Non-racemic mixture” is a mixture containing unequal amounts of enantiomers.

“Animal” refers to a living organism having sensation and the power of voluntary movement, and which requires for its existence oxygen and organic food. Examples include, without limitation, members of the human, equine, porcine, bovine, murine, canine, or feline species. In the case of a human, an “animal” may also be referred to as a “patient”.

“Mammal” refers to a warm-blooded vertebrate animal.

“Treating Huntington's disease” refers to:
(i) improving motor coordination in an animal having Huntington's disease; and/or
(ii) prolonging the survival of an animal having Huntington's disease.

In addition, “treating Huntington's disease” may optionally include:
(iii) preventing Huntington's disease from occurring in an animal that may be predisposed to Huntington's disease but has not yet been diagnosed as having it;
(iv) inhibiting or slowing Huntington's disease, e.g. arresting its development; and/or
(v) relieving Huntington's disease, e.g. causing its regression.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

This invention relates to a method for treating Huntington's disease comprising administering an effective amount of a NAALADase inhibitor to an animal or a mammal in need of such treatment.

This invention further relates to a pharmaceutical composition comprising:
(i) an effective amount of a NAALADase inhibitor for treating Huntington's disease; and
(ii) a pharmaceutically acceptable carrier.

The pharmaceutical composition may further comprise one or more pharmaceutical excipient(s), including one or more diluent(s), and/or wetting, emulsifying and/or pH buffering agent(s).

The inventive compounds and compositions may be administered locally or systemically by any means known to an ordinarily skilled artisan. For example, the inventive compounds and compositions may be administered orally, parenterally, by inhalation spray, topically, rectally, nasally, buccally, vaginally or via an implanted reservoir in dosage formulations containing conventional non-toxic pharmaceutically acceptable carriers, adjuvants and vehicles. The term parenteral as used herein includes subcutaneous, intravenous, intraarterial, intramuscular, intraperitoneal, intrathecal, intraventricular, intrasternal, intracranial or intraosseous injection and infusion techniques. The exact administration protocol will vary depending upon numerous factors including the age, body weight, general health, sex and diet of the patient; the determination of the exact administration protocol would be routine to an ordinarily skilled artisan. The inventive compounds and compositions may penetrate the blood-brain barrier when administered peripherally. Compounds and compositions that cannot penetrate the blood-brain barrier when administered peripherally may be administered intravenously or by other means recognized in the art. See, for example, U.S. Pat. Nos. 5,846,565; 5,651,986; and 5,626,862.

The inventive compounds and compositions may be administered by a single dose, multiple discrete doses or continuous infusion. In some embodiments pumps, such as subcutaneous pumps, are used for continuous infusion.

Dose levels on the order of about 0.001 to about 10,000 mg/kg/day of the active ingredient compound are useful in the inventive method. In some embodiments, the levels are about 0.1 to about 1,000 mg/kg, and in some embodiments the levels are about 1 to 100 mg/kg. The specific dose level for any particular patient will vary depending upon a variety of factors, including the activity and the possible toxicity of the specific compound employed; the age, body weight, general health, sex and diet of the patient; the time of administration; the rate of excretion; drug combination; the severity of the disease being treated; and the form of administration. Typically, in vitro dosage-effect results provide useful guidance on the proper doses for patient administration. Studies in animal models are also helpful. The considerations for determining the proper dose levels are well known in the art.

Any administration regimen well known to an ordinarily skilled artisan for regulating the timing and sequence of drug delivery can be used and repeated as desired to effect treatment in the inventive method. Such regimen may include pretreatment and/or co-administration with additional therapeutic agents.

The inventive method and composition may be used alone or in combination with one or more additional agent(s) for simultaneous, separate or sequential use.

The additional agent(s) may 'be any therapeutic agent(s) known to an ordinarily skilled artisan, including, without limitation, one or more compound(s) of formulas I-V.

The inventive compounds and compositions can be co-administered with one or more agent(s) either together in a single formulation, or separately in individual formulations designed for optimal release rates of their respective agent.

The inventive compounds and compositions may be administered before, during or after surgery or physical therapy.

NAALADase inhibitors that can be used in the inventive method and pharmaceutical composition include without limitation metallopeptidase inhibitors such as o-phenanthroline, metal chelators such as EGTA and EDTA, and peptide analogs such as quisqualic acid and β-NAAG.

There is evidence that the pathophysiology of Huntington's disease may involve glutamate excitotoxicity. Thus, in some embodiments the NAALADase inhibitor is one that is capable of reducing or preventing glutamate-induced excitotoxicity, thereby reducing or preventing neuronal damage or death resulting from such excitotoxicity. While the foregoing attributes are in some embodiments, the NAALADase inhibitors used in the inventive method and pharmaceutical composition may exert their therapeutic effects through other mechanisms of action.

In some embodiments, the NAALADase inhibitor is an acid containing a metal binding group.

In some embodiments, the NAALADase inhibitor is a compound of formula I 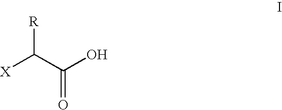
or an enantiomer or a pharmaceutically acceptable equivalent of said compound, wherein:
X is a moiety of formula II, III or IV 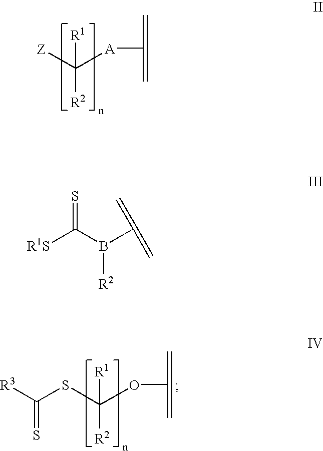
Z is SH, SO₃H, SO₂H, SOH, SO (NH) R⁴ or S (NHR⁴)₂R⁵;
B is N or CR⁶;
A is O, S, CR⁷R⁸ or (CR⁷R⁸)ₘS;
m and n are independently 0, 1, 2, 3 or 4;
R, R¹, R², R³, R⁴, R⁵, R⁶, R⁷ and R⁸ are independently hydrogen, C₁-C₉ alkyl, C₂-C₉ alkenyl, C₃-C₈ cycloalkyl, C₅-C₇ cycloalkenyl, Ar, hydroxy, carboxy, carbonyl, amino, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and
Ar is a carbocyclic or heterocyclic moiety, which is unsubstituted or substituted with one or more substituent(s);
provided that when X is a moiety of formula II and A is O, then n is 2, 3 or 4; when X is a moiety of formula II and A is S, then n is 2, 3 or 4; and when X is a moiety of formula II and A is (CR⁷R⁸)ₘS, then n is 0, 2, 3 or 4.

In some embodiments, X is a moiety of formula II; n is 0, 1, 2 or 3; Z is SH, SO₃H, SO₂H, SOH or S(NHR⁴)₂R⁵; and A is O, S or CR⁷R⁸.

In another embodiment, R is —(CH₂)₂COOH.

In a further embodiment, Z is SH.

In some embodiments, the NAALADase inhibitor is selected from:
2-(2-sulfanylethyl)pentanedioic acid;
3-(2-sulfanylethyl)-1,3,5-pentanetricarboxylic acid;
2-(2-sulfanylpropyl)pentanedioic acid;
2-(2-sulfanylbutyl)pentanedioic acid;
2-(2-sulfanyl-2-phenylethyl)pentanedioic acid;
2-(2-sulfanylhexyl)pentanedioic acid;
2-(2-sulfanyl-1-methylethyl)pentanedioic acid;
2-[1-(sulfanylmethyl)propyl)pentanedioic acid;
2-(3-sulfanylpentyl)pentanedioic acid;
2-(3-sulfanylpropyl)pentanedioic acid;
2-(3-sulfanyl-2-methylpropyl)pentanedioic acid;
2-(3-sulfanyl-2-phenylpropyl)pentanedioic acid;
2-(3-sulfanylbutyl)pentanedioic acid;
2-[3-sulfanyl-2-(phenylmethyl)propyl]pentanedioic acid;
2-[2-(sulfanylmethyl)butyl]pentanedioic acid;
2-[2-(sulfanylmethyl)pentyl]pentanedioic acid;
2-(3-sulfanyl-4-methylpentyl)pentanedioic acid; and
enantiomers and pharmaceutically acceptable equivalents.

### FORMULA V

In some embodiments, the NAALADase inhibitor is a compound of formula V 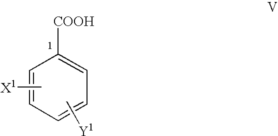
or an enantiomer or a pharmaceutically acceptable equivalent of said compound, wherein:
X¹ is —W-Z¹;
W is a bond or a linking group;
Z¹ is a terminal group; and
Y¹ is —COOH oriented meta or para relative to C-1.

Linking groups include, without limitation, divalent hydrocarbon chains, ethers, sulfides and amines, wherein the hydrocarbon chain, whether alone or part of the ether, sulfide or amine, may be saturated or unsaturated, straight or branched, open or closed, unsubstituted or substituted with one or more substituent(s), which in some embodiments are independently selected from C₁-C₆ alkoxy, C₂-C₆ alkenyloxy, phenoxy, benzyloxy, hydroxy, carboxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitro, nitroso, isonitroso, nitrosamino, imino, nitrilo, isonitrilo, nitrosimino, oxo, C₁-C₆ alkylthio, sulfamino, sulfamoyl, sulfeno, sulfhydryl, sulfinyl, sulfo, sulfonyl, sulfoxy, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, diselanyl, siloxy, silyl and silylene groups.

In some embodiments, W is a bond, —(CR⁹R¹⁰)ₙ—, —(CR⁹R¹⁰)ₙO(CR¹¹R¹²)ₘ—, —(CR⁹R¹⁰)ₙS(CR¹¹R¹²)ₘ— or —(CR⁹R¹⁰)ₙNR¹³(CR¹¹R¹²)ₘ—, wherein m and n are independently 0-9, and R⁹, R¹⁰, R¹¹, R¹² and R¹³ are independently hydrogen, C₁-C₆ alkyl, C₂-C₆ alkenyl, C₂-C₆ alkynyl, C₆-C₁₄ aryl, heteroaryl, C₆-C₁₄ carbocycle, heterocycle, halo, hydroxy, sulfhydryl, nitro, amino or C₁-C₆ alkoxy, and said alkyl, alkenyl, alkynyl, aryl, heteroaryl, carbocycle, heterocycle and alkoxy are independently unsubstituted or substituted with one or more substituent(s). In some embodiments, R⁹, R¹⁰, R¹¹, R¹² and R¹³ are each hydrogen and the total number of carbon atoms in W is 2-6.

In some embodiments, Z¹ is a metal binding group. In some embodiments, Z¹ is —COOH, —COR¹⁴, —OR¹⁴, —CF₃, —CN, —F, —Cl, —Br, —I, —NO, —NO₂, —C(O)(NR¹⁴OR¹⁵) —C(O)(NR¹⁴PO₃H₂), —C(O)(NR¹⁴R¹⁵), ═NOH, —NR¹⁴(P(O)(R¹⁵)OH), ═NR¹⁴, —N═NR¹⁴, 13 N(R¹⁴)CN, —NR¹⁴(CR¹⁵R¹⁶)_{P}COOH, —NR¹⁴(CO)NR¹⁵R¹⁶, —NR¹⁴(COOR¹⁵), —NR¹⁴(CO)R¹⁵, —NR¹⁴(OR¹⁵), —NR¹⁴R¹⁵, NR¹⁴(SO₂R¹⁵), —O(CO)R¹⁴, —OR¹⁴, —SO₂(OR¹⁴), —SO₂(NR¹⁴R¹⁵), —SO₂R¹⁴, —SO₃R¹⁴, —SNR¹⁴(OR¹⁵), —S(NR¹⁴R¹⁵), —SR¹⁴, —SSR¹⁴, —P(O)(OH)OR¹⁴, —P(O)(OH)R¹⁴ or —PR¹⁴R¹⁵, wherein p is 0-6, and R¹⁴, R¹⁵ and R¹⁶ are independently hydrogen, C₁-C₉ alkyl, C₂-C₉ alkenyl, C₂-C₉ alkynyl, C₆-C₁₄ aryl, heteroaryl, C₆-C₁₄ carbocycle, heterocycle, halo, hydroxy, sulfhydryl, nitro, amino or C₁-C₉ alkoxy, and said alkyl, alkenyl, alkynyl, aryl, heteroaryl, carbocycle, heterocycle and alkoxy are independently unsubstituted or substituted with one or more substituent(s). And in some embodiments, Z¹ is —NH(CR¹⁵R¹⁶)_{P}COOH, —PO(OH)OR¹⁴, —PO(OH)R¹⁴, —NR¹⁴(P(O)(R¹⁵)OH), —CON(R¹⁴)(OH) or —SH.

In some embodiments:
X¹ is —(CR⁹R¹⁰)ₙNH(CR¹¹R¹²)ₘCOOH, —PO(OH)OR¹⁴, —(CR⁹R¹⁰)ₙP(O)(OH)R¹⁴, —NH—(CR¹¹R¹²)ₘ-heteroaryl, —NH(P(O)(R¹⁵)OH), —(CR⁹R¹⁰)ₙNH(P(O)(OH)R¹⁵), —CON(R¹⁴)(OH), —(CR⁹CR¹⁰)ₙCON(R¹⁴)(OH), —(CR⁹R¹⁰)ₙSH, —O(CR¹¹R¹²)ₘSH, —SO₂NH-aryl, —N(C═O)—CH₂(C═O)-aryl, —SO₂NH-aryl, —N(C═O)—CH₂(C═O)-aryl, or —O-aryl wherein aryl in —O-aryl is substituted by at least one of nitro, carboxy or 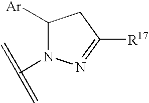
   wherein X¹ is oriented meta or para relative to C-1;
Ar is a carbocyclic or heterocyclic moiety, which is unsubstituted or substituted with one or more substituent(s);
m and n are independently 1-3, provided that when X¹ is —O(CR¹¹R¹²)ₘSH, then m is 2 or 3;
R⁹, R¹⁰, R¹¹, R¹², R¹⁴, R¹⁵ and R¹⁷ are independently hydrogen, C₁-C₆ alkyl, C₂-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituent(s); and
Y¹ is —COOH oriented meta or para relative to C-1.

In some embodiments, when X¹ is —PO(OH)OR¹⁴ or —(CR⁹R¹⁰)ₙP(O)(OH)OR¹⁴, then R¹⁴ is not H or methyl; when X¹ is —NH(P(O)(R¹⁵)OH or —(CR⁹R¹⁰)ₙNH(P(O)(OH)R¹⁵), then R¹⁵ is not benzyl unsubstituted or substituted with amino; and when X¹ is —CON(R¹⁴)(OH), then R¹⁴ is not H or methyl.

In another embodiment of formula V, X¹ is oriented meta relative to C-1, and Y¹ is oriented ortho relative to X¹ and para relative to C-1. In some embodiments, W is a bond, —(CH₂)ₙ—NH—(CH₂)ₘ— or —(CH₂)ₙ—; m is 1-3; n is 0-3; and Z¹ is —CO₂H, —NO₂, —NH₂, —SO₃H, halo, C₅-C₆ heteroaryl, carboxyphenylthio, or mono- or di-carboxyphenylsulfonyl.

In some embodiments, the NAALADase inhibitor is selected from:
2-[(4-carboxyphenyl)sulfonyl]-1,4-benzene-dicarboxylic acid;
2-[(2,5-dicarboxyphenyl)sulfonyl]-1,4-benzene-dicarboxylic acid;
1,2,4-benzenetricarboxylic acid;
2-[(2-carboxyphenyl)thio]-1,4-benzenedicarboxylic acid;
2-nitro-1,4-benzenedicarboxylic acid;
2-bromo-1,4-benzenedicarboxylic acid;
2-amino-1,4-benzenedicarboxylic acid;
2-sulfoterephthalic acid, monosodium salt;
2-carboxymethyl-1,4-benzenedicarboxylic acid;
2-[(2-furanylmethyl)-amino]-1,4-benzenedicarboxylic acid;
2-[(carboxymethyl)amino]-1,4-benzenedicarboxylic acid; and
enantiomers and pharmaceutically acceptable equivalents.

In another embodiment of formula V, X¹ is oriented ortho relative to C-1, and Y¹ is oriented para relative to X¹ and meta relative to C-1. In some embodiments, (1) when W is a bond, then Z¹ is —CO₂H, —OH, —NO₂, —C(O)(NHR¹⁵), —SR¹⁵, —COR¹⁵ or —NH(CH₂R¹⁵), and R¹⁵ is an aryl or a heteroaryl wherein said aryl and heteroaryl are independently unsubstituted or substituted with one or more alkyl, nitro or carboxy group(s); and (2) when W is —(CH₂)ₙ— and n is 1-3, then Z¹ is —SH.

In some embodiments, the NAALADase inhibitor is selected from:
4-(4-nitrobenzoyl)-1,3-benzenedicarboxylic acid;
4-[4-(2,4-dicarboxybenzoyl)phenoxy]-1,2-benzene-dicarboxylic acid;
4-[4-(2,4-dicarboxybenzoyl)phenoxy)-1,3-benzene-dicarboxylic acid;
4-[[(2,4,6-trimethylphenyl)amino]carbonyl)-1,3-benzenedicarboxylic acid;
4-nitro-1,3-benzenedicarboxylic acid;
4-[(1-naphthalenylamino)-carbonyl]-1,3-benzene-dicarboxylic acid;
1,2,4-benzenetricarboxylic acid;
4-[(2-carboxyphenyl)thio)-1,3-benzenedicarboxylic acid;
4-[3-[[3-(2,4-dicarboxyphenoxy)propyl]dithio]-propoxy]-1,3-benzenedicarboxylic acid;
4-hydroxy-1,3-benzenedicarboxylic acid;
4-[(2-furanylmethyl)amino]-1,3-benzenedicarboxylic acid;
4-(2-mercaptoethyl)-1,3-benzenedicarboxylic acid; and
enantiomers and pharmaceutically acceptable equivalents.

In another embodiment of formula V, X¹ is oriented meta relative to C-1, and Y¹ is oriented meta relative to X¹ and meta relative to C-1. In some embodiments, (1) when W is a bond, —(CH₂)ₙ— or —O(CH₂)ₘ— and m and n are independently 0-3, then Z¹ is —SO₃H, —NO₂, —NH₂, —CO₂H, —OH, —PO₃H, —CO(NHOH), —SH or an optionally substituted phenyl wherein one or more substituents are selected from nitro and carboxy; (2) when W is —(CH₂)ₙNH(CH₂)ₘ— and m and n are independently 0-3, then Z¹ is —CO₂H or C₅-C₆ heteroaryl; and (3) when W is —(CH₂)ₙ— wherein n is 0-3, then Z¹ is either (a) a heteroaryl that is unsubstituted or substituted with an aryl that is unsubstituted or substituted with one or more C₁-C₃ alkyl, halo, nitro or hydroxy group(s), or (b) then Z¹ is —SO₂(NHR¹⁶) or —NH(COR¹⁶), wherein R¹⁶ is an optionally substituted C₁-C₃ alkyl wherein one or more substituents are selected from oxo, phenyl, and substituted phenyl; and R¹⁶ may also be selected from an aryl that is unsubstituted or substituted with one or more nitro, amino, halo or hydroxy group(s).

In some embodiments the NAALADase inhibitor is selected from:
5-[4,5-dihydro-5-(4-hydroxyphenyl)-3-phenyl-1H-pyrazol-1-yl]-1,3-benzenedicarboxylic acid;
5-(4,5-dihydro-3-methyl-5-phenyl-1H-pyrazol-1-yl)-1,3-benzenedicarboxylic acid;
5-[[(4-chloro-3-nitrophenyl)amino]sulfonyl]-1,3-benzenedicarboxylic acid;
5-[[[4-chloro-3-[[3-(2-methoxyphenyl)-1,3-dioxopropyl)amino]phenyl]amino]sulfonyl-1,3-benzenedicarboxylic acid;
5-[[3-[4-(acetylamino)phenyl]-1,3-dioxopropyl]amino]-1,3-benzenedicarboxylic acid;
5-acetylamino-1,3-benzenedicarboxylic acid;
5-[[(1-hydroxy-2-naphthalenyl)carbonyl]-methylamino]-1,3-benzenedicarboxylic acid;
5-(4-carboxy-2-nitrophenoxy)-1,3-benzenedicarboxylic acid;
5-sulfo-1,3-benzenedicarboxylic acid;
5-nitro-1,3-benzenedicarboxylic acid;
5-amino-1,3-benzenedicarboxylic acid;
1,3,5-benzenetricarboxylic acid;
5-[[(3-amino-4-chlorophenyl)amino]sulfbonyl]-1,3-benzenedicarboxylic acid;
5-(3-mercaptopropoxy)-1,3-benzenedicarboxylic acid;
5-hydroxy-1,3-benzenedicarboxylic acid;
5-(2-mercaptoethoxy)-1,3-benzenedicarboxylic acid;
5-[(hydroxyamino)carbonyl]-1,3-benzenedicarboxylic acid;
5-phosphono-1,3-benzenedicarboxylic acid;
5-mercaptomethyl-1,3-benzenedicarboxylic acid;
5-phosphonomethyl-1,3-benzenedicarboxylic acid;
5-[[(carboxymethyl)amino]-methyl]-1,3-benzene-dicarboxylic acid;
5-[(carboxymethyl)amino]-1,3-benzenedicarboxylic acid;
5-[[(2-furanylmethyl)amino)-methyl)-1,3-benzene-dicarboxylic acid;
5-[2-(hydroxyamino)-2-oxoethyl]-1,3-benzene-dicarboxylic acid;
5-(2-mercaptoethyl)-1,3-benzenedicarboxylic acid; and
enantiomers and pharmaceutically acceptable equivalents.

Other NAALADase inhibitors are described in allowed U.S. patent application Ser. No. 09/378,443, now U.S. Pat. No. 6,313,159, issued Nov. 6, 2001, and U.S. patent application Ser. No. 09/438,970 filed Nov. 12, 1999, (corresponding to International Patent Application No. PCT/US00/30977 filed Nov. 13, 2000), now U.S. Pat. No. 6,348,464, issued Feb. 19, 2002, the entire contents of each of which publications, patents, and applications are herein incorporated by reference as though set forth herein in full.

Possible substituents of the compounds of formulas I-V include, without limitation, C₁-C₆ alkyl, C₂-C₆ alkenyl, C₂-C₆ alkynyl, C₁-C₆ alkoxy, C₂-C₆ alkenyloxy, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, disilanyl, siloxy, silyl, silylene and carbocyclic and heterocyclic moieties.

Carbocyclic moieties include alicyclic and aromatic structures. Examples of carbocyclic and heterocyclic moieties include, without limitation, phenyl, benzyl, naphthyl, indenyl, azulenyl, fluorenyl, anthracenyl, indolyl, isoindolyl, indolinyl, benzofuranyl, benzothiophenyl, indazolyl, benzimidazolyl, benzthiazolyl, tetrahydrofuranyl, tetrahydropyranyl, pyridyl, pyrrolyl, pyrrolidinyl, pyridinyl, pyrimidinyl, purinyl, quinolinyl, isoquinolinyl, tetrahydroquinolinyl, quinolizinyl, furyl, thiophenyl, imidazolyl, oxazolyl, benzoxazolyl, thiazolyl, isoxazolyl, isotriazolyl, oxadiazolyl, triazolyl, thiadiazolyl, pyridazinyl, pyrimidinyl, pyrazinyl, triazinyl, trithianyl, indolizinyl, pyrazolyl, pyrazolinyl, pyrazolidinyl, thienyl, tetrahydroisoquinolinyl, cinnolinyl, phthalazinyl, quinazolinyl, quinoxalinyl, naphthyridinyl, pteridinyl, carbazolyl, acridinyl, phenazinyl, phenothiazinyl, and phenoxazinyl.

All variables of formulas I-V are independently selected at each occurrence. For example, formula I may have two different CR¹R² moieties when X is a moiety of formula II and n is 2, with the first CR¹R² moiety being CH₂, and the second CR¹R² moiety being CH(CH₃).

The compounds of formulas I-V may possess one or more asymmetric carbon center(s) and, thus, may be capable of existing in the form of optical isomers as well as in the form of racemic or non-racemic mixtures of optical isomers. The optical isomers can be obtained by resolution of the racemic mixtures according to conventional processes well known in the art, for example by formation of diastereoisomeric salts by treatment with an optically active acid or base, and then separation of the mixture of diastereoisomers by crystallization followed by liberation of the optically active bases from these salts. Examples of optically active acids are tartaric, diacetyltartaric, dibenzoyltartaric, ditoluoyltartaric and camphorsulfonic acid. A different process for separation of optical isomers involves the use of a chiral chromatography column optimally chosen to maximize the separation of the enantiomers. Still another available method involves synthesis of covalent diastereoisomeric molecules, for example, esters, amides, acetals, ketals, and the like, by reacting compounds used in the inventive method and pharmaceutical composition with an optically active acid in an activated form, an optically active diol or an optically active isocyanate.

The synthesized diastereoisomers can be separated by conventional means such as chromatography, distillation, crystallization or sublimation, and then hydrolyzed to deliver the enantiomerically pure compound. In some embodiments, even without hydrolysis to the parent optically active drug, it is possible to dose the patient since the unhydrolyzed compound can behave as a prodrug. The optically active compounds can likewise be obtained by utilizing optically active starting materials.

It is understood that the compounds of formulas I-V encompass optical isomers as well as racemic and non- racemic mixtures.

Some of the NAALADase inhibitors used in the inventive method and pharmaceutical composition can be readily prepared by standard techniques of organic chemistry, utilizing the general synthetic pathways and examples depicted in U.S. Pat. Nos. 5,672,592, 5,795,877, 5,863,536, 5,880,112, 5,902,817, 5,962,521, 5,968,915, 6,025,344, 6,025,345, 6,028,216, 6,046,180, 6,054,444, 6,071,965, 6,121,252 and 6,265,609, allowed U.S. patent application Ser. No. 09/378,443, now U.S. Pat. No. 6,313,159, issued Nov. 6, 2001, copending U.S. patent application Ser. No. 09/438,970 filed Nov. 12, 1999 (corresponding to International Patent Application No. PCT/US00/30977 filed Nov. 13, 2000), now U.S. Pat. No. 6,348,464, issued Feb. 19, 2002, and International Publications Nos. WO 99/33849 and WO 00/01668, the entire contents of each of which patents, patent applications and publications are herein incorporated by reference, as though set forth herein in full.

Other NAALADase inhibitors may be available from commercial suppliers or can be readily prepared by an ordinarily skilled artisan using standard techniques such as those disclosed in U.S. Pat. No. 5,859,046, the entire contents of which reference are herein incorporated by reference as though set forth herein in full.

Yet other NAALADase inhibitors can be readily prepared by standard techniques of organic chemistry, utilizing the general synthetic pathways depicted below in SCHEMES I-IV.

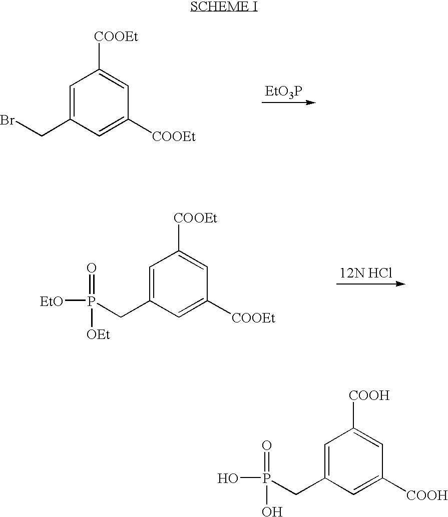

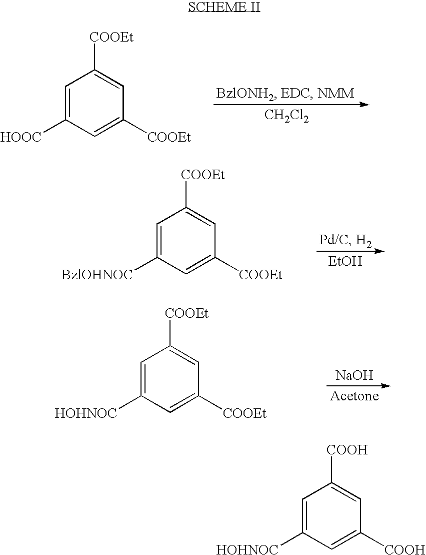

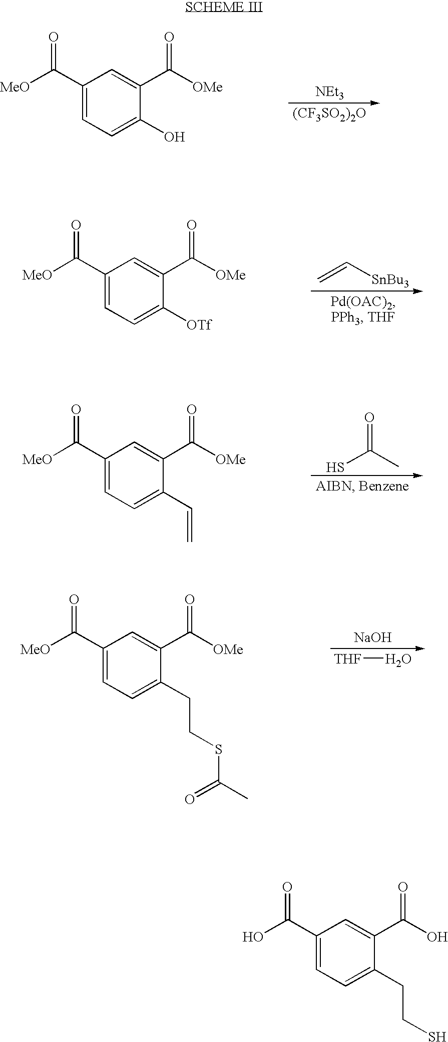

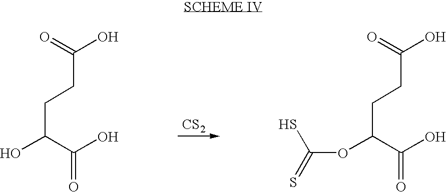

### EXAMPLES

The following examples are illustrative of this invention and are not intended to be limitations thereon. Unless otherwise indicated, all percentages are based upon 100% by weight of the final composition. Example 1

### Preparation of 5-Phosphonomethyl-1,3-Benzenedicarboxylic Acid (Scheme I)

### Diethyl 5-[(diethoxyphosphinyl)methyl]-1,3-benzenedicarboxylate

A solution of 5-bromomethyl-1,3-benzene-dicarboxylate (Collman et al., J. Am. Chem. Soc., 116(14)(1994) 6245-6251; 0.315 g, 1.0 mmol) in triethylphosphite (3.0 mL) was heated at 150° C. for 5 hours. The solvent was removed under reduced pressure and the residual oil was purified by chromatography to give 0.248 g of colorless oil: ¹H NMR (CDCl₃) δ 1.28 (t, 3H), 1.42 (t, 3H), 3.26 (d, 2H), 4.06 (q, 2H), 4.41 (q, 2H), 8.17 (s, 2H), 8.58 (s, 1H). TLC: R_{f} 0.10(EtOAc/Hexanes 1/1).

### 5-Phosphonomethyl-1,3-benzenedicarboxylic acid

A solution of diethyl 5-[(diethoxyphosphinyl) methyl]-1,3-benzenedicarboxylate (0.186 g, 0.5 mmol) in 12 N HCl (2.5 mL) was heated at 100° C. for 24 hours. The resulting precipitate was washed with water and dried under vacuum to give 0.057 g of white powder: ¹H NMR (D₂O) δ 3.11 (d, 2H), 7.93 (s, 2H), 8.19 (s, 1H). TLC: R_{f} 0.20 (EtOAc/Hexanes 1/1). Elemental analysis calculated for C₉H₇O₇P.H₂O: C, 38.86; H, 3.99. Found: C, 38.74; H, 4.08.

### Example 2

### preparation of 5-[(Hydroxyamino)Carbonyl]-1,3-Benzene-Dicarboxylic Acid (Scheme II)

### Diethyl 5-[[(phenylmethoxy)amino]carbonyl]-1,3-benzenedicarboxylate

To a solution of diethyl 1,3,5-benzenetricarboxylate (3.192 g, 20 mol) and O-benzylhydroxyamine hydrochloride (4.789 g, 19 mmol) in 40 mL were added N-methylmorpholine (2.2 mL, 20 mmol) and EDC (3.834 g, 20 mmol) at 0° C., and the mixture was stirred at room temperature for 20 hours. The solvent was removed by evaporator and the residue was dissolved in EtOAc (150 mL). The organic solution was washed with 1 N HCL (150 mL), washed with saturated aqueous NaHCO₃(50 mL), dried over Na₂SO₄, and concentrated to give white solid. This material was recrystallized from EtOAc to give 4.154 g of white powder: ¹H NMR (CDCl₃) δ 1.41(t, 6H), 4.40(q, 4H), 5.05(s, 2H), 7.3-7.5(m, 5H), 8.52(s, 2H), 8.76(s, 1H), 9.1(br, 1H). TLC: R_{f} 0.62 (EtOAc/Hexanes 1/1).

### Diethyl 5-(hydroxyamino)carbonyl]-1,3-benzenedicarboxylate

To a solution of diethyl 5-[[(phenylmethoxy)aminolcarbonyl]-1,3-benzenedicarboxylate (0.742 g, 2.0 mmol) in ethanol (10 mL) was added a suspension of Pd/C in ethanol (5 mL), and the mixture was shaken under hydrogen (50 psi) for 20 hours. The catalyst was removed by filtration through a pad of celite and the filtrate was concentrated to give white powder. This material was washed with ethanol (10 mL×2) and dried under vacuum to give 0.380 g of white powder: ¹H NMR (CD₃OD) δ 1.44 (t, 6H), 4.45 (q, 4H), 8.60 (s, 2H), 8.72 (s, 1H). TLC: R_{f} 0.20 (EtOAc/Hexanes 1/1).

### 5-[(Hydroxyamino)carbonyl]-1,3-benzene-dicarboxylic acid

To a solution of diethyl 5-[(hydroxyamino)carbonyl]-1,3-benzenedicarboxylate (0.281 g, 1.0 mmol) in acetone (5 mL) was added 1.0 N NaOH (5 mL) at room temperature, and the mixture was stirred at room temperature for 2 hours. The solvent was removed under reduced pressure and the residue was taken up with 1 N HCl (15 mL) to give white precipitate. This material was dried under vacuum to give 0.096 g of white solid: ¹H NMR (D₂O) δ 8.52 (s, 2H), 8.76 (s, 1H). Elemental analysis calculated for C₉H₇NO₆.H₂O: C, 44.45; H, 3.73; N, 5.76. Found: C, 44.47; H, 3.78; N, 5.74.

### Example 3

### Preparation of 4-(2-Mercaptoethyl)-1,3-Benzenedicarboxylic Acid (Scheme III)

### Dimethyl 4-trifluoromethanesulfonyloxy-1,3-benzenedicarboxylate

To a solution of dimethyl 4-hydroxy-isophthalate (0.850 g, 4.04 mmol) in CH₂Cl₂ (15 mL) were added triethylamine (0.6 mL, 4.3 mmol) and triflic anhydride (0.8 mL, 4.76 mmol) at 0° C., and the mixture was stirred at 0° C. for 18 hours. The solvent was evaporated and the residue was diluted with ether (30 mL). The organic solution was washed with 1 N HCl (30 mL×3), dried over MgSO₄, and concentrated to give 1.30 g of dark yellow oil (93% yield): ¹H NMR (CDCl₃) δ 3.97 (s, 3H), 4.00 (s, 3H), 7.4 (d, 1H), 8.3 (d, 1H), 8.74 (s, 1H).

### Dimethyl 4-ethenyl-1,3-benzenedicarboxylate

To a solution of dimethyl 4-trifluoromethanesulfonyl-oxy-1,3-benzenedicarboxylate (1.5 g, 4.38 mmol) in dioxane (50 mL) were added Pd(PPh₃)₄ (510 mg, 0.44 mmol), lithium chloride (1.3 g, 30.7 mmol) and tributyl(vinyl)tin (1.5 mL, 5.13 mmol) at room temperature. The mixture was heated at 100° C. for 5 hours. The reaction mixture was filtered and the filtrate was concentrated and passed through a column of silica gel (Hexanes/EtOAc=10:1) to give 1.1 g of colorless oil (84% yield): ¹H NMR: (CDCL₃) δ 3.92 (s, 3H), 3.93 (s, 3H), 5.45 (d, 1H), 5.73 (d, 1H), 7.49 (m, 1H), 7.66 (d, 1H), 8.13 (d, 1H), 8.53 (s, 1H).

### Dimethyl 4-[2-(acetylthio)ethyl]-1,3-benzenedicarboxylate

To a degassed solution of dimethyl 4-ethenyl-1,3-benzenedicarboxylate (415 mg, 1.88 mmol) in benzene (6 mL) were added AIBN (33 mg, 0.21 mmol) and thioacetic acid (0.27 mL, 3.78 mmol), and the mixture was refluxed for 5 hours. The reaction mixture was diluted with aqueous NaHCO₃ solution (15 mL) and extracted with EtOAc (15 mL). The organic layer was dried over MgSO₄ and concentrated. The residual material was purified by silica gel chromatography (hexanes/EtOAc=10:1) to give 0.150 g of colorless oil (27% yield): ¹H NMR (CDCl₃) δ 2.32 (s, 3H), 3.16 (t, 2H), 3.28 (t, 2H), 3.94 (s, 6H), 7.42 (d, 1H)), 8.09 (d, 1H), 8.58 (s, 1H).

### 4-(2-Mercaptoethyl)-1,3-benzenedicarboxylic acid

To a degassed solution of dimethyl 4-[2-(acetylthio)ethyl]-1,3-benzenedicarboxylate (0.130 g, 0.44 mmol) in THF (5 mL) was added a degassed solution of 5 N NaOH (5 mL). The reaction mixture was stirred under nitrogen overnight. The reaction mixture was diluted with H₂O (10 mL) and extracted with EtOAC (10 mL). The organic layer was dried over MgSO₄ and concentrated to give 0.045 g of white solid (45% yield): ¹H NMR (DMSO) δ 2.67 (t, 2H), 3.21 (t, 2H), 7.37 (d, 1H), 7.98 (d, 1H), 8.46 (s, 1H). ¹³C NMR (DMSO) δ 26.64, 40.60, 130.87, 132.05, 133.46, 133.81, 134.13, 148.53, 169.22, 170.20. Elemental analysis calculated for C₁₀H₁₀SO₄: C, 53.09; H, 4.45; S, 14.47. Found: C, 53.37; H, 4.87; S, 12.84. MS(FAB) 225.

### Example 4

### In Vitro Inhibition of NAALADase Activity

Various compounds used in the inventive method and pharmaceutical composition have been tested for in vitro inhibition of NAALADase activity. The experimental protocol and some results are set forth in U.S. Pat. Nos. 5,672,592, 5,795,877, 5,863,536, 5,880,112, 5,902,817, 5,962,521, 5,968,915, 6,025,344, 6,025,345, 6,028,216, 6,046,180, 6,054,444, 6,071,965, 6,121,252 and 6,265,609, allowed U.S. patent application Ser. No. 09/378,443, now U.S. Pat. No. 6,313,159, issued Nov. 6, 2001, copending U.S. patent application Ser. No. 09/438,970 filed Nov. 12, 1999 (corresponding to International Patent Application No. PCT/US00/30977 filed Nov. 13, 2000), U.S. Pat. No. 6,348,464, issued Feb. 19, 2002, and International Publications Nos. WO 99/33849 and WO 00/01668, the entire contents of each of which patents, patent applications, and publications are herein incorporated by reference, as though set forth herein in full.

Other results are provided below in TABLE I.

TABLE I
IN VITRO INHIBITION OF NAALADASE ACTIVITY

| Compound | IC₅₀ (nM) |
|---|---|
| 4-[4-(2,4-dicarboxybenzoyl)phenoxy]- | 1170 |
| 1,2-benzenedicarboxylic acid | |
| 2-[(4-carboxyphenyl)sulfonyl]-1,4- | 2370 |
| benzenedicarboxylic acid | |
| 2-[(2,5-dicarboxyphenyl)sulfonyl]-1,4- | 1870 |
| benzenedicarboxylic acid | |
| 4-[(2-carboxyphenyl)thio]-1,3- | 3980 |
| benzenedicarboxylic acid | |
| 2-[(2-carboxyphenyl)thio]-1,4- | 572 |
| benzenedicarboxylic acid | |
| 4-[3-[[3-(2,4-dicarboxyphenoxy)- | 3750 |
| propyl]-dithio]propoxy]-1,3- | |
| benzenedicarboxylic acid | |
| 5-(3-mercaptopropoxy)-1,3- | 3300 |
| benzenedicarboxylic acid | |
| 5-(2-mercaptoethoxy)-1,3- | 14500 |
| benzenedicarboxylic acid | |
| 5-[(hydroxyamino)-carbonyl]-1,3- | 1000 |
| benzenedicarboxylic acid | |
| 5-phosphono-1,3-benzenedicarboxylic | 14000 |
| acid | |
| 5-mercaptomethyl-1,3- | 6500 |
| benzenedicarboxylic acid | |
| 5-phosphonomethyl-1,3- | 3100 |
| benzenedicarboxylic acid | |
| 5-[(carboxymethyl)amino]-1,3- | 100000 |
| benzenedicarboxylic acid | |
| 5-[[(2-furanylmethyl)amino]methyl]- | 50000 |
| 1,3-benzenedicarboxylic acid | |
| 2-carboxymethyl-1,4- | 9000 |
| benzenedicarboxylic acid | |
| 5-[2-(hydroxyamino)-2-oxoethyl]-1,3- | 12000 |
| benzenedicarboxylic acid | |
| 4-(2-mercaptoethyl)-1,3- | 116 |
| benzenedicarboxylic acid | |
| 5-(2-mercaptoethyl)-1,3- | 5100 |
| benzenedicarboxylic acid | |

### Example 5

### Neuroprotective Effect of NAALADase Inhibitors in Transgenic Mouse Model of Huntington's Disease

Behavioral Testing (Rotarod)

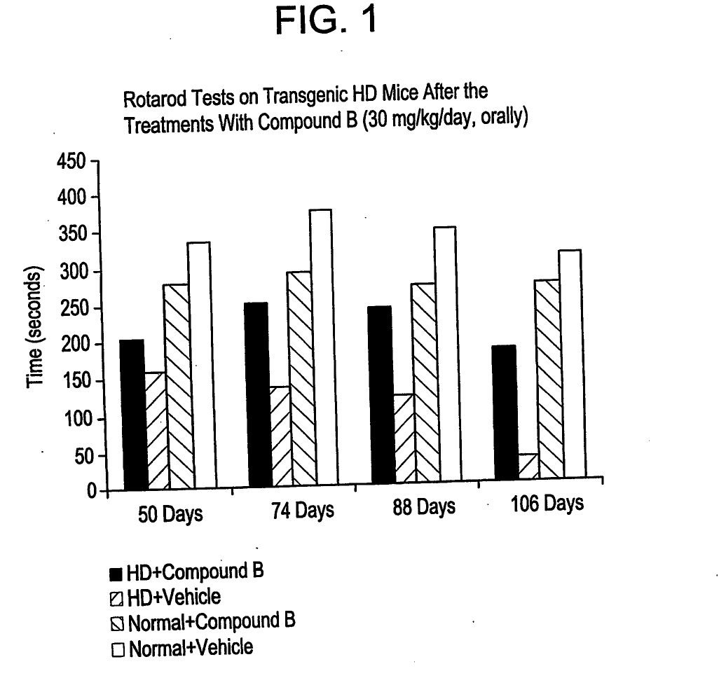

Transgenic HD mice of the N171-82Q strain and non-transgenic littermates were treated with NAALADase inhibitor Compound B (30 mg/kg) or a vehicle from 10 weeks of age. The mice were placed on a rotating rod (“rotarod”). The length of time at which the mouse fell off the rotarod was recorded as a measure of motor coordination. FIG. 1 shows that transgenic HD mice treated with Compound B stayed longer on the rotarod than similar transgenic HD mice treated with a vehicle. The treatment with Compound B had no effect on the rotarod performance of normal non-HD mice.

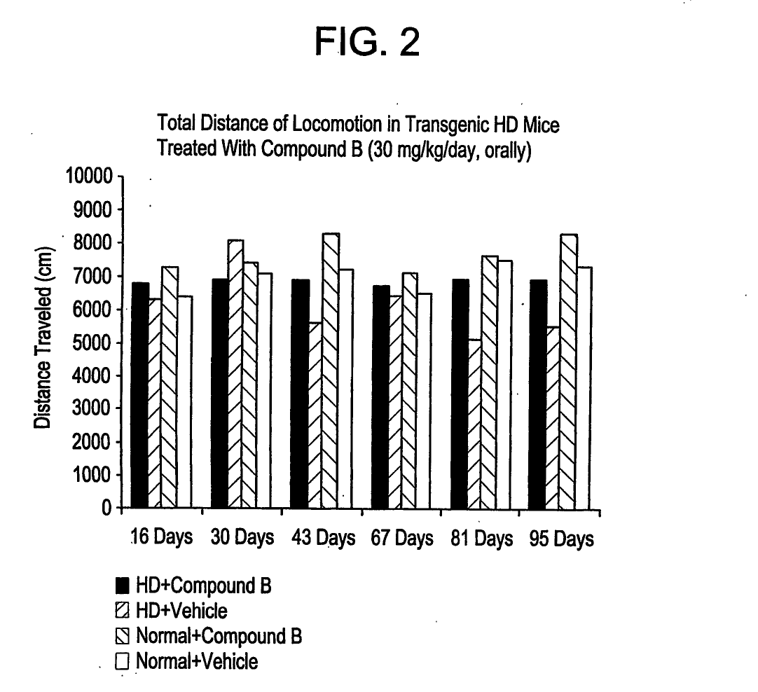

The total distance traveled by the mice was also recorded as a measure of overall locomotion. FIG. 2 shows that while the vehicle treated HD mice demonstrated the lowest mean locomotor score, the treatment with NAALADase inhibitor had no apparent effect on overall locomotion.

Survival

The effects of Compound B and vehicle on the survival of transgenic HD mice (N171-82Q) were evaluated. Thirteen mice (six male and seven female) were assigned to the Compound B treatment group, and fourteen mice (six male and eight female) were assigned to the vehicle treatment group. Treatment was continued until all the mice died.

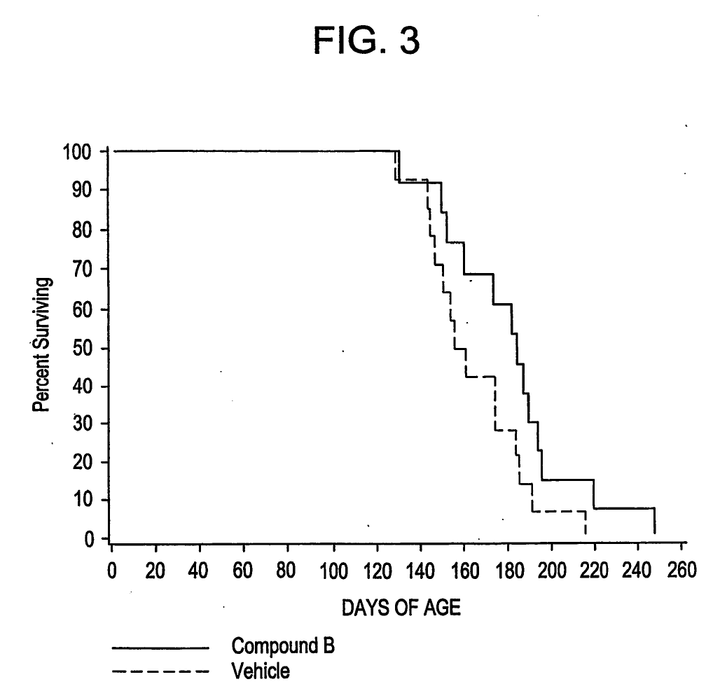

FIG. 3 shows the survival distributions over time by treatment group. The median survival time is 184 days for the Compound B treatment group, and 158.5 days for the vehicle treatment group. Although the Compound B treatment group had a longer median survival time than the vehicle treatment group, the difference is not statistically significant (p-value=0.07).

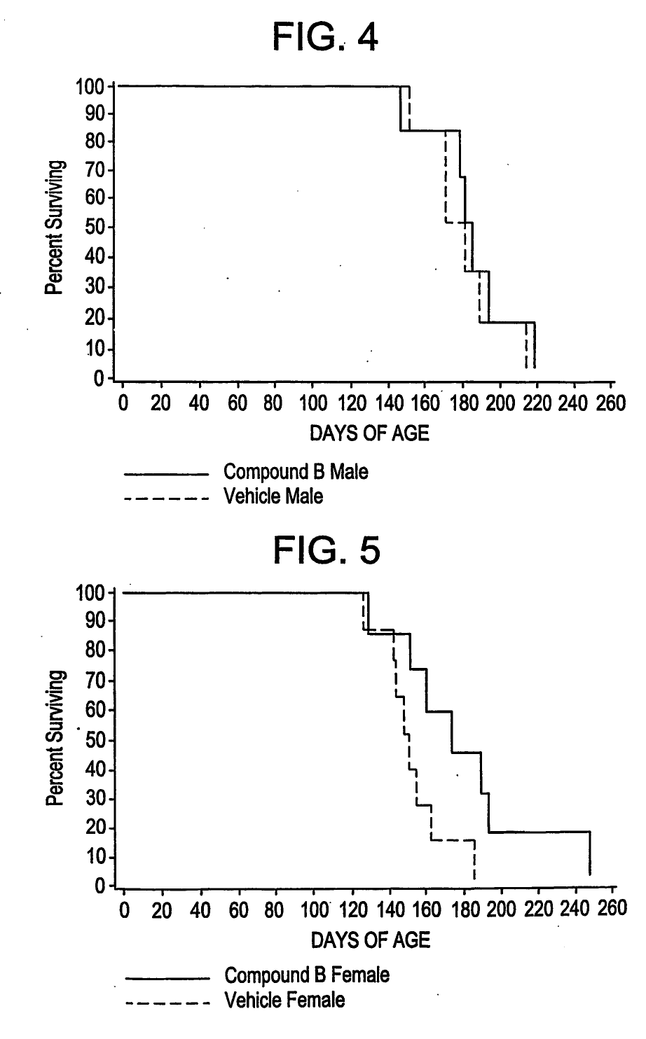

FIGS. 4 and 5 show the survival distributions over time by treatment group and sex. When analyzing the results specific to sex, female mice treated with Compound B had significantly prolonged survival time (p-value 0.03) compared to their vehicle treated counterparts. Within the vehicle treatment group, the males have better survival times than the females although this trend was not observed in the Compound B treatment group. The data suggest that sex may influence survival distributions over time.

All publications, patents and patent applications identified above are herein incorporated by reference, as though set forth herein in full.

The invention being thus described, it will be apparent to those skilled in the art that the same may be varied in many ways without departing from the spirit and scope of the invention. Such variations are included within the scope of the following claims.

1. A method for treating Huntington's disease comprising administering an effective amount of a NAALADase inhibitor to a mammal in need of such treatment.

2. The method of claim 1, wherein the NAALADase inhibitor is an acid containing a metal binding group.

3. The method of claim 1, wherein the NAALADase inhibitor is a compound of formula I 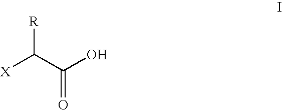
or an enantiomer or a pharmaceutically acceptable equivalent of said compound, wherein:
X is a moiety of formula II, III or IV 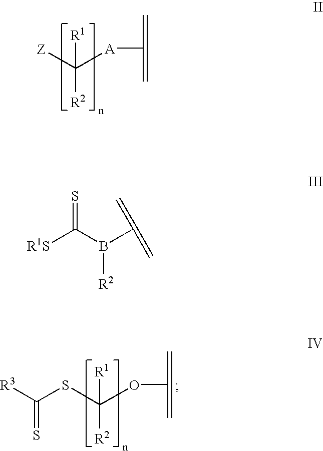
Z is SH, SO₃H, SO₂H, SOH, SO(NH)R⁴ or S(NHR⁴)₂R⁵;
B is N or CR⁶;
A is O, S, CR⁷R⁸ or (CR⁷R⁸)ₘS;
m and n are independently 0, 1, 2, 3 or 4;
R, R¹, R², R³, R⁴, R⁵, R⁶, R⁷ and R⁸ are independently hydrogen, C₁-C₉ alkyl, C₂-C₉ alkenyl, C₃-C₆ cycloalkyl, C₅-C₇ cycloalkenyl, Ar, hydroxy, carboxy, carbonyl, amino, cyano, isocyano, nitro, sulfonyl, sulfoxy, thio, thiocarbonyl, thiocyano, formanilido, thioformamido, sulfhydryl, halo, haloalkyl, trifluoromethyl or oxy, wherein said alkyl, alkenyl, cycloalkyl and cycloalkenyl are independently unsubstituted or substituted with one or more substituent(s); and
Ar is a carbocyclic or heterocyclic moiety, which is unsubstituted or substituted with one or more substituent (s);
provided that when X is a moiety of formula II and A is O, then n is 2, 3 or 4; when X is a moiety of formula II and A is S, then n is 2, 3 or 4; and when X is a moiety of formula II and A is (CR⁷R⁸)ₘS, then n is 0, 2, 3 or 4.

4. The method of claim 3, wherein:
X is a moiety of formula II;
n is 0, 1, 2 or 3;
Z is SH, SO₃H, SO₂H, SOH or S(NHR⁴)₂R⁵; and
A is O, S or CR⁷R⁸.

5. The method of claim 4, wherein Z is SH.

6. The method of claim 5, wherein R is —(CH₂)₂COOH.

7. The method of claim 1, wherein the NAALADase inhibitor is selected from:
2-(2-sulfanylethyl)pentanedioic acid;
3-(2-sulfanylethyl)-1,3,5-pentanetricarboxylic acid;
2-(2-sulfanylpropyl)pentanedioic acid;
2-(2-sulfanylbutyl)pentanedioic acid;
2-(2-sulfanyl-2-phenylethyl)pentanedioic acid;
2-(2-sulfanylhexyl)pentanedioic acid;
2-(2-sulfanyl-1-methylethyl)pentanedioic acid;
2-[1-(sulfanylmethyl)propyl]pentanedioic acid;
2-(3-sulfanylpentyl)pentanedioic acid;
2-(3-sulfanylpropyl)pentanedioic acid;
2-(3-sulfanyl-2-methylpropyl)pentanedioic acid;
2-(3-sulfanyl-2-phenylpropyl)pentanedioic acid;
2-(3-sulfanylbutyl)pentanedioic acid;
2-[3-sulfanyl-2-(phenylmethyl)propyl]pentanedioic acid;
2-[2-(sulfanylmethyl)butyl]pentanedioic acid;
2-[2-(sulfanylmethyl)pentyl]pentanedioic acid;
2-(3-sulfanyl-4-methylpentyl)pentanedioic acid; and
enantiomers and pharmaceutically acceptable equivalents.

8. The method of claim 1, wherein the NAALADase inhibitor is a compound of formula V 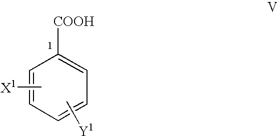
or an enantiomer or a pharmaceutically acceptable equivalent of said compound, wherein:
X¹ is —W-Z¹;
W is a bond or a linking group;
Z¹ is a terminal group; and
Y¹ is —COOH oriented meta or para relative to C-1.

9. The method of claim 8, wherein:
X¹ is —(CR⁹R¹⁰)ₙNH(CR¹¹R¹²)ₘCOOH, —PO (OH)OR¹⁴, —(CR⁹R¹⁰)ₙP(O)(OH)R¹⁴, —NH—(CR¹¹R¹²)ₘ-heteroaryl, —NH(P(O)(R¹⁵)OH), —(CR⁹R¹⁰)ₙNH(P(O)(OH)R¹⁵), —CON(R¹⁴)(OH), —(CR⁹CR¹⁰)ₙCON(R¹⁴)(OH), —(CR⁹R¹⁰)ₙSH, —O(CR¹¹R¹²)ₘSH, —SO₂NH-aryl, —N(C═O)—CH₂(C═O)-aryl, —SO₂NH-aryl, —N(C═O)—CH₂(C═O)-aryl or —O-aryl, wherein aryl in —O-aryl is substituted by at least one of nitro, carboxy or 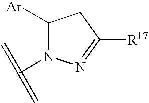
wherein X¹ is oriented meta or para relative to C-1;
Ar is a carbocyclic or heterocyclic moiety, which is unsubstituted or substituted with one or more substituent (s);
m and n are independently 1-3, provided that when X¹ is —O(CR¹¹R¹²)ₘSH, then m is 2 or 3;
R⁹, R¹⁰, R¹¹, R¹², R¹⁴, R¹⁵ and R¹⁷ are independently hydrogen, C₁-C₆ alkyl, C₂-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituent(s); and
Y¹ is —COOH oriented meta or para relative to C-1.

10. The method of claim 8, wherein X¹ is oriented ortho relative to C-1, and Y¹is oriented para relative to X¹ and meta relative to C-1.

11. The method of claim 10, wherein W is a bond, and Z¹ is —CO₂H, —OH, —NO₂, —C(O)(NHR¹⁵), —SR¹⁵, —COR¹⁵ or —NH(CH₂R¹⁵), and R¹⁵ is an aryl or a heteroaryl wherein said aryl and heteroaryl are independently unsubstituted or substituted with one or more alkyl, nitro or carboxy group(s).

12. The method of claim 10, wherein W is —(CH₂)ₙ— and n is 1-3, and Z¹ is —SH.

13. The method of claim 8, wherein the linking groups are selected from divalent hydrocarbon chains, ethers, sulfides and amines, wherein the hydrocarbon chains, whether alone or part of ethers, sulfides, and/or amines, may be saturated or unsaturated, straight or branched, open or closed, unsubstituted or substituted with one or more substituents.

14. The method of claim 13, wherein the one or more substituents are independently selected from C₁-C₆ alkoxy, C₂-C₆ alkenyloxy, phenoxy, benzyloxy, hydroxy, carboxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitro, nitroso, isonitroso, nitrosamino, imino, nitrilo, isonitrilo, nitrosimino, oxo, C₁-C₆ alkylthio, sulfamino, sulfamoyl, sulfeno, sulfhydryl, sulfinyl, sulfo, sulfonyl, sulfoxy, thiocarboxy, thiocyano, isothiocyano, thioformamido, halo, haloalkyl, chlorosyl, chloryl, perchloryl, trifluoromethyl, iodosyl, iodyl, phosphino, phosphinyl, phospho, phosphono, arsino, selanyl, diselanyl, siloxy, silyl and silylene groups.

15. The method of claim 8, wherein W is a bond, —(CR⁹R¹⁰)ₙ—, —(CR⁹R¹⁰)ₙO(CR¹¹R¹²)ₘ—, —(CR⁹R¹⁰)ₙS (CR¹¹R¹²)ₘ— or —(CR⁹R¹⁰)ₙNR¹³(CR¹¹R¹²)ₘ—, wherein m and n are independently 0-9, and R⁹, R¹⁰, R¹¹, R¹² and R¹³ are independently hydrogen, C₁-C₆ alkyl, C₂-C₆ alkenyl, C₂-C₆ alkynyl, C₆-C₁₄ aryl, heteroaryl, C₆-C₁₄ carbocycle, heterocycle, halo, hydroxy, sulfhydryl, nitro, amino or C₁-C₆ alkoxy, and said alkyl, alkenyl, alkynyl, aryl, heteroaryl, carbocycle, heterocycle and alkoxy are independently unsubstituted or substituted with one or more substituents.

16. The method of claim 15, wherein R⁹, R¹⁰, R¹¹, R¹² and R¹³ are each hydrogen and the total number of carbon atoms in W is 2-6.

17. The method of claim 8, wherein Z¹ is a metal binding group.

18. The method of claim 8, wherein Z¹ is —COOH, —COR¹⁴, —OR¹⁴, —CF₃, —CN, —F, —Cl, —Br, —I, —NO, —NO₂, —C(O)(NR¹⁴OR¹⁵), —C(O)(NR¹⁴PO₃H₂), —C(O)(NR¹⁴R¹⁵), ═NOH, —NR¹⁴ (P(O)(R¹⁵)OH), ═NR¹⁴, —N═NR¹⁴, —N(R¹⁴)CN, —NR¹⁴(CR¹⁵R¹⁶)_{P}COOH, NR¹⁴(CO)NR¹⁵R¹⁶, —NR¹⁴(COOR¹⁵), —NR¹⁴(CO)R¹⁵, —NR¹⁴(OR¹⁵), —NR¹⁴R¹⁵, —NR¹⁴(SO₂R¹⁵), —O(CO)R¹⁴, —OR¹⁴, —SO₂(OR¹⁴), —SO₂(NR¹⁴R¹⁵), —SO₂R¹⁴, —SO₃R¹⁴, —SN¹⁴(OR¹⁵), —S(NR¹⁴R¹⁵), —SR¹⁴, —SSR¹⁴, —P(O)(OH)OR¹⁴, —P(O)(OH)R¹⁴ or —PR¹⁴R¹⁵, wherein p is 0-6, and R¹⁴, R¹⁵ and R¹⁶ are independently hydrogen, C₁-C₉ alkyl, C₂-C₉ alkenyl, C₂-C₉ alkynyl, C₆-C₁₄ aryl, heteroaryl, C₆-C₁₄ carbocycle, heterocycle, halo, hydroxy, sulfhydryl, nitro, amino or C₁-C₉ alkoxy, and said alkyl, alkenyl, alkynyl, aryl, heteroaryl, carbocycle, heterocycle and alkoxy are independently unsubstituted or substituted with one or more substituents.

19. The method of claim 18, wherein Z¹ is —NH(CR¹⁵R¹⁶)_{P}COOH, —PO(OH)OR¹⁴, —PO(OH)R¹⁴, —NR¹⁴(P(O)(R¹⁵)OH), —CON(R¹⁴)(OH) or —SH.

20. The method of claim 8, wherein
X¹ is —(CR⁹R¹⁰)ₙNH(CR¹¹R¹²)ₘCOOH, —PO(OH)OR¹⁴, —(CR⁹R¹⁰)ₙP(O)(OH)R¹⁴, —NH—(CR¹¹R¹²)ₘ-heteroaryl, —NH(P(O)(R¹⁵)OH), —(CR⁹R¹⁰)ₙNH(P(O)(OH)R¹⁵), —CON(R¹⁴)(OH), —(CR⁹CR¹⁰)ₙCON(R¹⁴)(OH), —(CR⁹R¹⁰)ₙSH, —O(CR¹¹R¹²)ₘH, —SO₂NH-aryl, —N(C═O)—CH₂(C═O)-aryl, —SO₂NH-aryl, —N(C═O)—CH₂(C═O)-aryl, or —O-aryl wherein aryl in —O-aryl is substituted by at least one of nitro, carboxy or 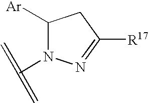
wherein X¹ is oriented meta or para relative to C-1;
Ar is a carbocyclic or heterocyclic moiety, which is unsubstituted or substituted with one or more substituent(s);
m and n are independently 1-3, provided that when X¹ is —O(CR¹¹-R¹²)ₘSH, then m is 2 or 3;
R⁹, R¹⁰, R¹¹, R¹², R¹⁴, R¹⁵ and R¹⁷ are independently hydrogen, C₁-C₆ alkyl, C₂-C₆ alkenyl, C₂-C₆ alkynyl, aryl, heteroaryl, carbocycle, heterocycle, halo, hydroxy, sulfhydryl, nitro, amino or C₁-C₆ alkoxy, wherein said alkyl, alkenyl, alkynyl, aryl, heteroaryl, carbocycle, heterocycle and alkoxy are independently unsubstituted or substituted with one or more substituents; and
Y¹ is —COOH oriented meta or para relative to C-1.

21. The method of claim 20, wherein X¹ is —PO(OH)OR¹⁴ or —(CR⁹R¹⁰))P(O)(OH)OR¹⁴, and R¹⁴ is not H or methyl.

22. The method of claim 20, wherein X¹ is —NH(P(O)(R¹⁵)OH or —(CR⁹R¹⁰)ₙNH(P(O)(OH)R¹⁵), and R¹⁵ is not benzyl unsubstituted or substituted with amino.

23. The method of claim 20, wherein X¹ is —CON(R¹⁴)(OH), and R¹⁴ is not H or methyl

24. The method of claim 8, wherein X¹ is oriented meta relative to C-1, and Y¹ is oriented ortho relative to X¹ and para relative to C-1.

25. The method of claim 24, wherein W is a bond, —(CH₂)ₙ—NH—(CH₂)ₘ— or —(CH₂)ₙ—; m is 1-3; n is 0-3; and Z¹ is —CO₂H, —NO₂, —NH₂, —SO₃H, halo, C₅-C₆ heteroaryl, carboxyphenylthio, or mono- or di-carboxyphenylsulfonyl.

26. The method of claim 8, wherein X¹ is oriented meta relative to C-1, and Y¹is oriented meta relative to X¹ and meta relative to C-1.

27. The method of claim 26, wherein W is a bond, —(CH₂)ₙ— or —O(CH₂)ₘ— and m and n are independently 0-3, and Z¹ is —SO₃H, —NO₂, —NH₂, —CO₂H, —OH, —PO₃H, —CO(NHOH), —SH or an optionally substituted phenyl wherein one or more substituents are selected from nitro and carboxy.

28. The method of claim 26, wherein W is —(CH₂)ₙNH(CH₂)ₘ— and m and n are independently 0-3, and Z¹ is —CO₂H or C₅-C₆ heteroaryl.

29. The method of claim 26, wherein W is —(CH₂)ₙ— wherein n is 0-3, and (a) Z¹ is a heteroaryl that is unsubstituted or substituted with an aryl that is unsubstituted or substituted with one or more C₁-C₃ alkyl, halo, nitro or hydroxy group(s), or (b) Z¹ is —SO₂(NHR¹⁶) or —NH(COR¹⁶), wherein R¹⁶ is an optionally substituted C₁-C₃ alkyl wherein one or more substituents are selected from oxo, phenyl, and substituted phenyl; and R¹⁶ may also be selected from an aryl that is unsubstituted or substituted with one or more nitro, amino, halo or hydroxy group(s).

30. The method of claim 1, wherein the NAALADase inhibitor is selected from:
2-[(4-carboxyphenyl)sulfonyl]-1,4-benzene-dicarboxylic acid;
2-[(2,5-dicarboxyphenyl)sulfonyl]-1,4-benzene-dicarboxylic acid;
1,2,4-benzenetricarboxylic acid;
2-[(2-carboxyphenyl)thio]-1,4-benzenedicarboxylic acid;
2-nitro-1,4-benzenedicarboxylic acid;
2-bromo-1,4-benzenedicarboxylic acid;
2-amino-1,4-benzenedicarboxylic acid;
2-sulfoterephthalic acid, monosodium salt;
2-carboxymethyl-1,4-benzenedicarboxylic acid;
2-[(2-furanylmethyl)-amino]-1,4-benzenedicarboxylic acid;
2-[(carboxymethyl)amino]-1,4-benzenedicarboxylic acid;
4-(4-nitrobenzoyl)-1,3-benzenedicarboxylic acid;
4-[4-(2,4-dicarboxybenzoyl)phenoxyl-1,2-benzene-dicarboxylic acid;
4-[4-(2,4-dicarboxybenzoyl)phenoxy]-1,3-benzene-dicarboxylic acid;
4-[[(2,4,6-trimethylphenyl)amino]carbonyl]-1,3-benzenedicarboxylic acid;
4-nitro-1,3-benzenedicarboxylic acid;
4-[(1-naphthalenylamino)-carbonyl]-1,3-benzene-dicarboxylic acid;
1,2,4-benzenetricarboxylic acid;
4-[(2-carboxyphenyl)thiol-1,3-benzenedicarboxylic acid;
4-[3-[[3-(2,4-dicarboxyphenoxy)propyl]dithio]-propoxy]-1,3-benzenedicarboxylic acid;
4-hydroxy-1,3-benzenedicarboxylic acid;
4-[(2-furanylmethyl)amino]-1,3-benzenedicarboxylic acid;
4-(2-mercaptoethyl)-1,3-benzenedicarboxylic acid;
5-[4,5-dihydro-5-(4-hydroxyphenyl)-3-phenyl-1H-pyrazol-1-yl]-1,3-benzenedicarboxylic acid;
5-(4,5-dihydro-3-methyl-5-phenyl-1H-pyrazol-1-yl)-1,3-benzenedicarboxylic acid;
5-[[(4-chloro-3-nitrophenyl)amino]sulfonyl]-1,3-benzenedicarboxylic acid;
5-[[[4-chloro-3-[[3-(2-methoxyphenyl)-1,3-dioxopropyl]aminolphenyl]amino]sulfonyl-1,3-benzenedicarboxylic acid;
5-[[3-[4-(acetylamino)phenyl)-1,3-dioxopropyl]amino]-1,3-benzenedicarboxylic acid;
5-acetylamino-1,3-benzenedicarboxylic acid;
5-[[(1-hydroxy-2-naphthalenyl)carbonyl]-methylamino]-1,3-benzenedicarboxylic acid;
5-(4-carboxy-2-nitrophenoxy)-1,3-benzenedicarboxylic acid;
5-sulfo-1,3-benzenedicarboxylic acid;
5-nitro-1,3-benzenedicarboxylic acid;
5-amino-1,3-benzenedicarboxylic acid;
1,3,5-benzenetricarboxylic acid;
5-[[(3-amino-4-chlorophenyl)amino]sulfonyl]-1,3-benzenedicarboxylic acid;
5-(3-mercaptopropoxy)-1,3-benzenedicarboxylic acid;
5-hydroxy-1,3-benzenedicarboxylic acid;
5-(2-mercaptoethoxy)-1,3-benzenedicarboxylic acid;
5-[(hydroxyamino)carbonyl]-1,3-benzenedicarboxylic acid;
5-phosphono-1,3-benzenedicarboxylic acid;
5-mercaptomethyl-1,3-benzenedicarboxylic acid;
5-phosphonomethyl-1,3-benzenedicarboxylic acid;
5-[[(carboxymethyl)amino)-methyl]-1,3-benzene-dicarboxylic acid;
5-[(carboxymethyl)amino]-1,3-benzenedicarboxylic acid;
5-[[(2-furanylmethyl)amino]-methyl]-1,3-benzene-dicarboxylic acid;
5-[2-(hydroxyamino)-2-oxoethyl]-1,3-benzene-dicarboxylic acid;
5-(2-mercaptoethyl)-1,3-benzenedicarboxylic acid; and
enantiomers and pharmaceutically acceptable equivalents.

31. A method for treating Huntington's disease comprising administering an effective amount of a compound selected from:
2-[(4-carboxyphenyl)sulfonyl]-1,4-benzene-dicarboxylic acid;
2-[(2,5-dicarboxyphenyl)sulfonyl]-1,4-benzene-dicarboxylic acid;
1,2,4-benzenetricarboxylic acid;
2-[(2-carboxyphenyl)thiol-1,4-benzenedicarboxylic acid;
2-nitro-1,4-benzenedicarboxylic acid;
2-bromo-1,4-benzenedicarboxylic acid;
2-amino-1,4-benzenedicarboxylic acid;
2-sulfoterephthalic acid, monosodium salt;
2-carboxymethyl-1,4-benzenedicarboxylic acid;
2-[(2-furanylmethyl)-amino]-1,4-benzenedicarboxylic acid;
2-[(carboxymethyl)amino]-1,4-benzenedicarboxylic acid;
4-(4-nitrobenzoyl)-1,3-benzenedicarboxylic acid;
4-[4-(2,4-dicarboxybenzoyl)phenoxy]-1,2-benzene-dicarboxylic acid;
4-[[(2,4,6-trimethylphenyl)amino]carbonyl]-1,3-benzenedicarboxylic acid;
4-nitro-1,3-benzenedicarboxylic acid;
4-[(1-naphthalenylamino)-carbonyl]-1,3-benzene-dicarboxylic acid;
1,2,4-benzenetricarboxylic acid;
4-[(2-carboxyphenyl)thiol-1,3-benzenedicarboxylic acid;
4-[3-[[3-(2,4-dicarboxyphenoxy)propyl]dithio]-propoxy]-1,3-benzenedicarboxylic acid;
4-hydroxy-1,3-benzenedicarboxylic acid;
4-[(2-furanylmethyl)amino]-1,3-benzenedicarboxylic acid;
4-(2-mercaptoethyl)-1,3-benzenedicarboxylic acid;
5-[4,5-dihydro-5-(4-hydroxyphenyl)-3-phenyl-1H-pyrazol-1-yl]-1,3-benzenedicarboxylic acid;
5-(4,5-dihydro-3-methyl-5-phenyl-1H-pyrazol-1-yl)-1,3-benzenedicarboxylic acid;
5-[[(4-chloro-3-nitrophenyl)amino]sulfonyl]-1,3-benzenedicarboxylic acid;
5-[[[4-chloro-3-[[3-(2-methoxyphenyl)-1,3-dioxopropyl]amino)phenyl]amino]sulfonyl-1,3-benzenedicarboxylic acid;
5-[[3-[4-(acetylamino)phenyl]-1,3-dioxopropyl]amino]-1,3-benzenedicarboxylic acid;
5-acetylamino-1,3-benzenedicarboxylic acid;
5-[[(1-hydroxy-2-naphthalenyl)carbonyl]-methylamino]-1,3-benzenedicarboxylic acid;
5-(4-carboxy-2-nitrophenoxy)-1,3-benzenedicarboxylic acid;
5-sulfo-1,3-benzenedicarboxylic acid;
5-nitro-1,3-benzenedicarboxylic acid;
5-amino-1,3-benzenedicarboxylic acid;
1,3,5-benzenetricarboxylic acid;
5-[[(3-amino-4-chlorophenyl)amino]sulfonyl]-1,3-benzenedicarboxylic acid;
5-(3-mercaptopropoxy)-1,3-benzenedicarboxylic acid;
5-hydroxy-1,3-benzenedicarboxylic acid;
5-(2-mercaptoethoxy)-1,3-benzenedicarboxylic acid;
5-[(hydroxyamino)carbonyl]-1,3-benzenedicarboxylic acid;
5-phosphono-1,3-benzenedicarboxylic acid;
5-mercaptomethyl-1,3-benzenedicarboxylic acid;
5-phosphonomethyl-1,3-benzenedicarboxylic acid;
5-[[(carboxymethyl)amino]-methyl]-1,3-benzene-dicarboxylic acid;
5-[(carboxymethyl)amino)-1,3-benzenedicarboxylic acid;
5-[[(2-furanylmethyl)amino]-methyl]-1,3-benzene-dicarboxylic acid;
5-[2-(hydroxyamino)-2-oxoethyl]-1,3-benzene-dicarboxylic acid;
5-(2-mercaptoethyl)-1,3-benzenedicarboxylic acid; and
enantiomers and pharmaceutically acceptable equivalents.

32. A pharmaceutical composition comprising:
(i) an effective amount of a NAALADase inhibitor for treating Huntington's disease; and
(ii) a pharmaceutically acceptable carrier.

33. A method of making a pharmaceutical composition comprising mixing an effective amount of a NAALADase inhibitor for treating Huntington's disease and a pharmaceutically acceptable carrier.
